(19) United States
(12) Patent Application Publication
(10) Pub. No.: US 2024/0182925 A1
(43) Pub. Date: Jun. 6, 2024

(54) INSULIN GENE THERAPY TO TREAT DIABETES

(71) Applicant: Wisconsin Alumni Research Foundation, Madison, WI (US)

(72) Inventors: Tausif ALAM, Madison, WI (US); Hans SOLLINGER, Madison, WI (US)

(21) Appl. No.: 18/551,110

(22) PCT Filed: Mar. 16, 2022
(86) PCT No.: PCT/US2022/020626

§ 371 Date: Sep. 18, 2023

Related U.S. Application Data
(60) Provisional application No. 63/161,495, filed on Mar. 16, 2021.
(60) Provisional application No. 63/165,310, filed on Mar. 24, 2021.

(51) Int. Cl. C12N 15/86; A61K 48/00; A61P 3/10; C07K 14/62

(52) U.S. Cl. CPC C12N 15/86; A61K 48/0058; A61P 3/10; C07K 14/62; C12N 2750/14143; C12N 2830/002; C12N 2830/008; C12N 2830/50

(57) ABSTRACT

The present invention provides recombinant adeno-associated virus serotype 8 (AAV8) virus particles that are designed to deliver insulin-encoding polynucleotides to the liver. Also provided are the nucleic acid constructs carried by the virus particles, methods for producing the AAV8 virus particles, and methods of using the AAV8 virus particles to control blood glucose levels in a mammal.

### CROSS-REFERENCE TO RELATED APPLICATIONS

This application claims priority to U.S. Provisional Application No. 63/161,495 filed on Mar. 16, 2021, and to U.S. Provisional Application No. 63/165,310 filed on Mar. 24, 2021, the contents of which are incorporated by reference in their entireties.

### STATEMENT REGARDING FEDERALLY SPONSORED RESEARCH

N/A

### SEQUENCE LISTING

A Sequence Listing accompanies this application and is submitted as an ASCII text file of the sequence listing named “960296_04288_ST25.txt” which is 19,010 bytes in size and was created on Mar. 14, 2022. The sequence listing is electronically submitted via EFS-Web with the application and is incorporated herein by reference in its entirety.

### BACKGROUND

Insulin is normally produced in and secreted by the beta cells of the islets of Langerhans in the pancreas. Mature insulin is a protein having two polypeptide chains, A and B, held together by disulfide bonds. The glucose-responsive release of insulin from the beta cells is a complex event involving gene expression, posttranslational modification, and secretion. The initial protein product is an insulin precursor referred to as preproinsulin, a single polypeptide chain having an N-terminal signal sequence and an intervening sequence, the C-peptide, between the B and A chains. The signal sequence is cleaved during transport from the rough endoplasmic reticulum to form proinsulin. Proinsulin is packaged into secretory granules along with specific enzymes required for its processing. Proinsulin folds into a specific three-dimensional structure, forming disulfide bonds. Mature insulin results from removal of the C-peptide. In beta cells, this function is catalyzed by endopeptidases that recognize the specific amino acid sequences at the junction of the B chain and the C peptide (i.e., the B-C junction) and at the junction of the C chain and the A peptide (i.e., the C-A junction). Mature insulin, which is stored in secretory granules, is released in response to elevated blood glucose levels. The detailed mechanism of insulin release is not completely understood, but the process involves migration and fusion of the secretory granules with the plasma membrane prior to release.

In normally functioning beta cells, insulin production and release are affected by the glycolytic flux. Glucokinase and glucose transporter 2 (GLUT-2) are two proteins that are believed to be involved in sensing changes in the glucose concentration in beta cells. A reduction in GLUT-2, which is involved in glucose transport, is correlated with decreased expression of insulin and loss of glucokinase activity and causes a rapid inhibition of insulin expression.

Autoimmune destruction of pancreatic beta cells causes insulin-dependent diabetes mellitus or type I diabetes. Due to the partial or complete loss of beta cells, little or no insulin is secreted by the pancreas in this disease. Most cells, with the exception of brain cells, require insulin for the uptake of glucose. Inadequate insulin production causes reduced glucose uptake and elevated blood glucose levels. Both reduced glucose uptake and high blood glucose levels are associated with very serious health problems. In fact, without proper treatment, diabetes can be fatal.

One conventional treatment for diabetes involves periodic administration of injectable exogenous insulin. This method has extended the life expectancy of millions of people with the disease. However, blood glucose levels must be carefully monitored to ensure that the individual receives an appropriate amount of insulin. Too much insulin can cause blood glucose levels to drop to dangerously low levels. Too little insulin will result in elevated blood glucose levels. Even with careful monitoring of blood glucose levels, control of diet, and insulin injections, the health of the vast majority of individuals with diabetes is adversely impacted in some way.

Replacement of beta cell function, which would allow insulin to be secreted by cells in response to glucose levels in the microenvironment, would eliminate the need for insulin injections. One approach to replacing beta cell function is pancreas transplantation, which has met with some success. However, the usefulness of this therapy is limited by the shortage of donor organs and the need for lifelong immunosuppression. Thus, there is a continued need in the art for improved treatments for type 1 diabetes.

### SUMMARY

The present invention provides nucleic acid constructions AAV8 vector, AAV8 virus particles and methods for delivering polynucleotides encoding insulin to liver cells in order to express insulin in a subject in need thereof.

In a first aspect, the disclosure provides nucleic acid constructs designed to deliver insulin-encoding polynucleotides to the liver for the treatment of type1 diabetes. The constructs comprise: (a) a 5′ inverted terminal repeat (ITR); (b) a promoter enhancer; (c) a single glucose inducible regulatory element (GIRE); (d) a liver-specific promoter; (e) a translational enhancer; (f) a polynucleotide encoding insulin with a modified peptidase site; (g) an albumin 3′ untranslated region (UTR); and (h) a 3′ ITR. The constructs contain only one GIRE.

In a second aspect, the present invention provides host cells transduced with the constructs described herein.

In a third aspect, the present invention provides recombinant adeno-associated virus serotype 8 (AAV8) virus particles comprising the constructs described herein.

In a fourth aspect, the present invention provides packaging cell lines for producing the virus particles described herein.

In a fifth aspect, the present invention provides methods for controlling blood glucose levels in a mammal by administering the recombinant AAV8 virus particles described herein. In these methods, the mammal's glucose levels are controlled by glucose-regulated synthesis of insulin from the nucleic acid construct.

In a sixth aspect, the present invention provides methods of producing an AAV8 virus particle. The methods comprise (a) transducing a host cell with: (i) a plasmid comprising SEQ ID NO:1, (ii) a packaging plasmid, and (iii) a helper plasmid; (b) collecting the supernatant and cells from the culture; and (c) isolating the virus.

### BRIEF DESCRIPTION OF THE DRAWINGS

FIG. 1 shows schematic depictions of a set of new insulin-encoding constructs that were produced in an effort to increase insulin expression for gene therapy. Various elements of the constructs are color coded as defined in the legend.

FIG. 2 is a graph showing the therapeutic response to sc.C19-AAV8 in a mouse model. A group of five streptozotocin (STZ) treated diabetic mice (blood glucose level at or >500 mg/dl) were treated with sc.C19-AAV8 at a dose of 8×10¹² vg/kg and were monitored for correction of hyperglycemia.

FIG. 3 is a graph showing the therapeutic dose response to C36-AAV8. A group of three STZ treated diabetic mice (blood glucose level at or >500 mg/dl) were treated with C36-AAV8 at the three indicated doses and were monitored for correction of hyperglycemia.

FIG. 4 is a photograph showing the CsCl equilibrium density gradient profile of C36-AAV8. The thick major band (second from the bottom) contains functionally active C36-AAV8.

FIG. 5 is a photograph showing the CsCl equilibrium density gradient profile of sc.C19-AAV8. Multiple, relatively faint bands were observed. The second band from the bottom contains functionally active sc.C16-AAV8.

FIG. 6 shows a vector map of C36-AAV8 labeling the relevant parts fo the construct.

### DETAILED DESCRIPTION

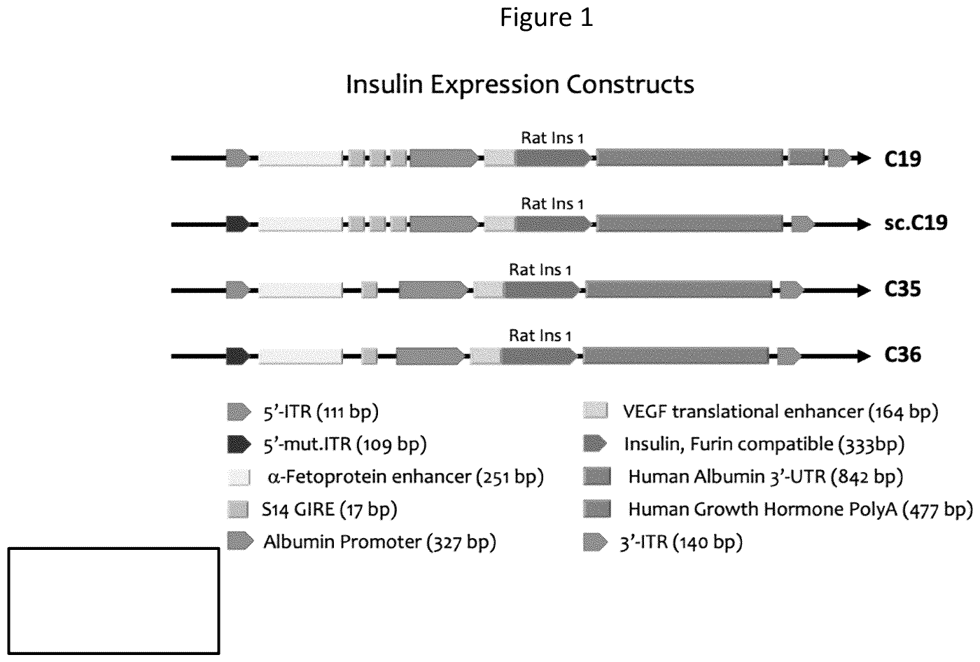

The present disclosure is directed to an improved insulin gene therapy vector that utilizes adeno-associated virus serotype 8 (AAV8) gene delivery. As is described in the Examples, the inventors tested four different vector constructs (depicted schematically in FIG. 1) that each contained inverted terminal repeats, a fetoprotein enhancer sequence, one or three glucose inducible regulatory elements (GIRE), an albumin promoter, a VEGF translational enhancer, a polynucleotide encoding insulin, and a human albumin UTR sequence. The inventors had thought that constructs containing multiple GIREs would be more effective as insulin gene therapies than constructs containing a single GIRE because they expected them to drive more insulin expression at lower glucose levels (i.e., make the construct more glucose-sensitive). However, unexpectedly, they found that constructs containing a single GIRE are more efficacious at controlling hyperglycemia in diabetic mice than constructs containing three GIREs, and that construct length can cause insulin expression problems. Surprisingly, the use of a single GIRE resulted in an approximately 80-fold improvement in both yield and insulin production in cells over the use of three GIREs. Specifically, use of a single GIRE increased viral production by about 10-fold and increased biological efficacy by about 8-fold over the use of three GIREs.

The gene therapies of the present invention are designed to deliver insulin-encoding polynucleotides to the liver. These gene therapies will enable the hepatocytes (i.e., liver cells) of patients to synthesize and secrete insulin in response to changing glucose levels. A single intravenous treatment with these gene therapies is adequate to control hyperglycemia in mice for a substantial portion of their lives. Thus, these gene therapies are expected to provide long-term benefits to patients with type I diabetes.

Hepatocytes have long been a preferred source of surrogate beta cells. They are long-lived cells that can be used as robust protein factories. Hepatocytes receive an extensive blood supply and are accessible by blood-borne particles, such as viruses. Moreover, hepatocytes express glucose-sensing molecules that are nearly identical to those expressed in the pancreas (i.e., GLUT2, glucokinase). Thus, these cells have an intrinsic ability to respond to changes in blood glucose concentration.

Viral vectors are the most efficient systems to deliver polynucleotides into a cell. For use in a gene therapy, wild-type virus is genetically modified to be nonpathogenic and unable to replicate in the absence of additional plasmids. Adeno-associated virus (AAV) vectors are the preferred and most commonly used viral vectors for both research and clinical applications. AAV is a helper-dependent parvovirus containing a single-stranded, linear DNA genome. These viruses are non-pathogenic and can infect both dividing and non-dividing cells. One disadvantage of AAV is its relatively small packaging capacity (4.7 Kb), which limits the size of the transgenes that can be inserted into it. Fortunately, the pre-proinsulin gene is small enough that even when the gene is accompanied by multiple regulatory elements in a complex expression cassette, this size limitation is unlikely to become an issue.

One of the major hurdles facing AAV gene delivery is the host immune response. Most people (>70%) are exposed to wild-type AAV early in life and are positive for anti-AAV antibodies for one or more AVV serotypes. Unfortunately, because recombinant AAV capsids are very similar to the wild-type AAV capsid, preexisting neutralizing antibodies against wild-type AAV can limit recombinant AAV transduction in these people. Furthermore, even among individuals that lack preexisting anti-AAV antibodies, re-administration of the same AAV vector (i.e., for treatments in which a second administration is required) is not likely to be effective due to the presence of anti-AAV antibodies induced by the first administration. Thus, the improved efficacy of the viral vectors described herein should also improve their safety for use as gene therapies, i.e., by allowing a lower viral dose to be used and thereby minimizing the immune response to the vector. As a result, the constructs of the present invention may allow for longer persistence of the insulin gene, producing longer lasting results.

### Nucleic Acid Constructs

The present invention provides nucleic acid constructs comprising: (a) a 5′ inverted terminal repeat (ITR); (b) a promoter enhancer; (c) a glucose inducible regulatory element (GIRE); (d) a liver-specific promoter; (e) a translational enhancer; (f) a polynucleotide encoding insulin with a modified peptidase site; (g) an albumin 3′ untranslated region (UTR); and (h) a 3′ ITR. As is described above, the inventors made the surprising discovery that constructs containing only a single GIRE are more effective than constructs containing three GIREs for use in insulin gene therapies. Thus, the nucleic acid constructs of the present invention contain only one GIRE.

In some embodiments, the constructs consist essentially of components (a)-(h). By “consist essentially of” we mean that constructs of the present invention consist of components (a)-(h) and possibly other regulatory elements necessary for construct function. For example, the constructs may include additional sequences that facilitate the addition or removal of functional elements (e.g., restriction sites) or additional sequences that are necessary for the replication of the constructs but do not alter the constructs' function (i.e., expression of insulin). In some embodiments, components (a)-(h) are present in the vector in the following 5′ to 3′ order: (a), (b), (c), (d), (e), (f), (g), and (h).

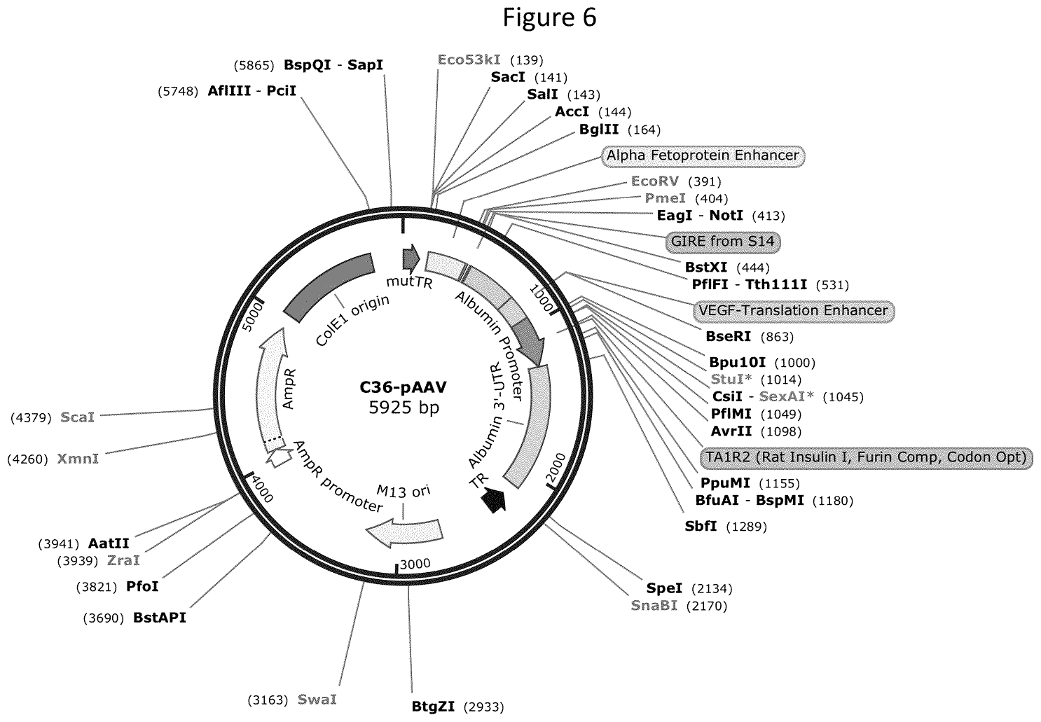

In some embodiments, the nucleic acid construct is SEQ ID NO:1, i.e., the C36-AAV8 construct that was tested by the inventors in the Examples and is depicted as a vector map in FIG. 6. SEQ ID NO:1 comprises all the components that are required in the constructs of the present invention (i.e., components (a)-(h)). However, in other embodiments, linking and nonessential sequences within SEQ ID NO:1 (e.g., sequences that are useful for cloning) may be substituted with other sequences that provide similar functions.

As used herein, the term “nucleic acid construct” refers to a recombinant polynucleotide, i.e., a polynucleotide that was formed artificially by combining at least two polynucleotide components from different sources (natural or synthetic). For example, a construct may comprise the coding region of one gene operably linked to a promoter that (1) is associated with another gene found within the same genome, (2) from the genome of a different species, or (3) is synthetic. Constructs can be generated using conventional recombinant DNA methods. Nucleic acid constructs may be part of a vector. When referring to a nucleic acid molecule alone (as opposed to a viral particle), the term “vector” is used herein to refer to a nucleic acid molecule capable of transporting another nucleic acid to which it is linked. Some vectors are capable of autonomous replication in a host cell into which they are introduced, while other vectors can be integrated into the genome of a host cell such that they are replicated along with the host genome.

The constructs of the present invention comprise 5′ and 3′ inverted terminal repeats. “Inverted terminal repeats (ITRs)” are inverted repeats that are found at the ends of the adeno-associated virus (AAV) genome. They contain several cis-acting elements that are involved in the initiation of viral DNA replication, as well as binding motifs for cellular transcription factors. Thus, the inclusion of ITRs in the constructs of the present invention allows the constructs to be incorporated into an adeno-associated viral particle and replicated for viral production. In some embodiments, the 5′ ITR is SEQ ID NO:2, i.e., the mutated 5′ ITR (mutTR) utilized in the C36-AAV8 construct (SEQ ID NO:1). In some embodiments, the 3′ ITR is SEQ ID NO:9, i.e., the mutated 3′ ITR (TR) utilized in the C36-AAV8 construct (SEQ ID NO:1).

The constructs of the present invention also comprise a promoter enhancer. As used herein, the term “promoter enhancer” is used to refer to a sequence that promotes transcription of a functionally linked polynucleotide by enhancing promoter function. Any promoter enhancer that enhances the activity of the liver-specific promoter included in the construct may be used with the present invention. In some embodiments, the promoter enhancer is an alpha-fetoprotein enhancer. The alpha-fetoprotein enhancer increases the activity of the albumin promoter by increasing the binding of RNA polymerase, resulting in an increase in mRNA and protein production. Endogenous transcription factors present in liver cells interact with the native alpha-fetoprotein enhancer, allowing it to activate the native albumin and alpha-fetoprotein promoters. However, in the fully developed liver, the alpha-fetoprotein enhancer is repressed. Accordingly, in some embodiments, the region associated with this repression is not included in the alpha-fetoprotein enhancer sequence, allowing enhancer activity to persist in fully developed liver cells. In certain embodiments, the promoter enhancer is the alpha-fetoprotein enhancer of SEQ ID NO:3, i.e., the promoter enhancer utilized in the C36-AAV8 construct (SEQ ID NO:1).

To achieve glucose-responsive insulin release in non-beta cells, the constructs of the present invention also comprise a single glucose inducible regulatory element. “Glucose inducible regulatory elements (GIREs)” are glucose-responsive DNA motifs found in the promoter region of glucose-inducible genes, such as LPK, S14, fatty acid synthase, and acetyl-CoA carboxylase. GIREs are composed of two tandem repeats of the nucleotide sequence 5′-CACGTG (known as E boxes), separated by five base pairs. When a specific transcription factor, i.e., carbohydrate response element-binding protein (ChREBP), recognizes the E box sequences, it results in glucose-responsive control of transcription. As is demonstrated in the Examples, GIREs provide transcriptional regulation of insulin in hepatocytes in response to physiologically relevant glucose concentrations. Thus, inclusion of a single GIRE in the constructs of the present invention makes them glucose responsive. Suitable GIREs are also described in U.S. Pat. Nos. 7,425,443 and 6,933,133, which are both hereby incorporated by reference. In some embodiments, the single GIRE is SEQ ID NO:4, i.e., the GIRE from the promoter of the liver protein S14, which was utilized in the C36-AAV8 construct (SEQ ID NO:1). The present invention found it surprising that only one GIRE provides improved AAV virus titers and insulin expression.

The constructs of the present invention also comprise a liver-specific promoter. As used herein, the term “promoter” refers to a DNA sequence that regulates the transcription of a polynucleotide. Typically, a promoter is a regulatory region that is capable of binding RNA polymerase and initiating transcription of a downstream sequence. However, a promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA segments. It is understood by those skilled in the art that different promoters may direct the expression of a gene in different tissues or cell types, at different stages of development, or in response to different environmental conditions. Promoters that cause a gene to be expressed in most cell types at most times are commonly referred to as “constitutive promoters”, whereas promoters that allow for controlled expression of a gene (e.g., under particular conditions or in the presence of a particular molecule) are referred to as “inducible promoters”. A promoter is “operably linked” to a polynucleotide if the promoter is connected to the polynucleotide such that it may affect transcription of the polynucleotide

As used herein, the term “liver-specific promoter” is used to refer to a promoter that only drives expression of an operably linked polynucleotide in liver cells (i.e., hepatocytes). A liver-specific promoter is used with the constructs of the present invention to ensure that production of insulin is restricted only to liver cells when the constructs are utilized in a gene therapy. Any constitutively active liver-specific promoter that drives sustained, moderate- to high-level transcription can be used in the constructs of the present invention. One example of such a promoter is alpha 1-antitrypsin inhibitor (Blood 84:3394-404, 1994). In some embodiments, the liver-specific promoter is an albumin promoter. For example, in some embodiments, the albumin promoter is the rat albumin promoter (which was produced as described in Transplantation 74:1781, 2002 and characterized as described in Mol Cell Biol 7:2425, 1987). In certain embodiments, the liver-specific promoter is the rat albumin promoter of SEQ ID NO:5, i.e., the liver-specific promoter utilized in the C36-AAV8 construct (SEQ ID NO:1). However, albumin promoters from other species, such as humans, are expected to confer similar properties to the constructs and may also be used in the constructs of the present invention.

The constructs of the present invention also comprise a translational enhancer. As used herein, the term “translational enhancer” refers to a sequence that promotes translation of a functionally linked polynucleotide. In some embodiments, the translational enhancer is a vascular endothelial growth factor (VEGF) translational enhancer. The VEGF translational enhancer enhances translation by acting as a ribosomal entry site. Thus, its presence results in greater insulin protein production from a given amount of insulin mRNA. In certain embodiments, the translational enhancer is the VEGF translational enhancer of SEQ ID NO:6, i.e., the translational enhancer utilized in the C36-AAV8 construct (SEQ ID NO:1). However, any translational enhancer that increases the translation of insulin from the constructs may be used with the present invention. Other suitable translational enhancers include, for example, the untranslated 5′ leader sequence (called Omega) of the tobacco mosaic virus RNA and the Kozak sequence.

The constructs of the present invention also comprise a polynucleotide encoding insulin with a modified peptidase site. Insulin is synthesized as a single-chain precursor (i.e., pre-proinsulin) consisting of A and B chains separated by a connecting (C)-peptide as well as a signal peptide. Cleavage of the signal peptide from pre-proinsulin yields proinsulin which undergoes further maturation through the proteolytic action of two specific prohormone convertases, PC1/3 and PC2. Thus, for an insulin gene therapy to be successful, the insulin-encoding polynucleotide must undergo appropriate post-translational processing. The prohormone convertases are only expressed in beta cells and other cells with the insulin secretory pathway (e.g., pituitary cells and intestinal K cells). Thus, an unmodified insulin-encoding polynucleotide that is expressed in the liver will produce unprocessed proinsulin, which has biological activity that is approximately 100-fold less than mature insulin, because the specific enzymes necessary for maturation are absent in liver cells. To overcome this challenge, the peptidase site in the insulin-encoding polynucleotide used with the present invention is modified to make it cleavable by a liver protease (e.g., furin), allowing insulin to be processed into the mature product in the liver.

In some embodiments, the insulin protein encoded by the construct can be processed by the endogenous liver protease furin. In some embodiments, the polynucleotide encoding insulin has at least 80%, at least 85%, at least 90%, at least 95%, at least 98%, or at least 99% sequence identity to SEQ ID NO:7. In certain embodiments, the polynucleotide encoding insulin is SEQ ID NO:7. SEQ ID NO:7 is a codon optimized polynucleotide that encodes a version of the rat insulin I protein that was modified (i.e., from KTRR to RTKR) at the junction of the B and C peptides of proinsulin to make this junction recognizable by furin. To make the human insulin protein furin-compatible, it is modified at two junctions of proinsulin that are proteolytically processed: at the B and C peptide junction (i.e., from KTRR to RTKR) and at the C and A peptide junction (i.e., from LQKR to RQKR). These modifications are described in the following publications: Hum Gene Ther 7:71, 1996; J Biol Chem 269:6241, 1994, incorporated by reference in its entirety. Suitable human insulin cDNA constructs can be constructed from what is known in the art. The constructs of the present invention may be used to treat human or non-human animals. In some embodiments, a construct encoding rat insulin is used to treat other species of animals (e.g., dogs and cats) in which rat insulin is tolerated. Alternatively, one may wish to substitute insulin sequences from other animals when treating those animals. Thus, the polynucleotide encoding insulin may be from any insulin-producing animal. In some embodiments, to ensure that no immune reaction to the insulin protein occurs when diabetic animals are treated using the gene therapy, a species-specific insulin gene is used. As used herein, the term “insulin gene” refers to any polynucleotide encoding insulin. This term includes simplified polynucleotide sequences such as complementary DNA (cDNA) which can further be engineered (e.g., to contain a furin-cleavage site) and is not limited to naturally occurring gene sequences. In any case, one can use published gene sequences (e.g., those for cats and dogs (J Biol Chem 258 2357-2363, 1983)) to generate primers to amplify the insulin coding sequence from cDNA preparations made from isolated pancreatic RNA from the desired species using standard molecular biology techniques, or can synthesize the cDNA chemically. For example, the polynucleotide encoding insulin may be derived from any of the genes listed in Table 1, which come from rat, cat, dog, and human. In some embodiments, the polynucleotide encoding insulin has at least 80%, at least 85%, at least 90%, at least 95%, at least 98%, or at least 99% sequence identity (including 100%) to the coding region of the insulin sequences listing in Table 1. As described above for human insulin, polynucleotides encoding non-human insulin should be modified such that they encode an insulin protein in which the recognition sites for peptidases found in β-cells are changed into sites that are recognized by proteases found in the liver (e.g., furin). For example, but not limited to, a suitable human insulin polypeptide sequence can be found in the NCBI protein bank (e.g., SEQ ID NO:14; NCBI Reference Sequence: NP_001278826.1) and can be modified to include a furin cleavage site (e.g., modifications are described in the following publications: Hum Gene Ther 7:71, 1996; J Biol Chem 269:6241, 1994, which is hereby incorporated by reference in its entirety).

“Percentage of sequence identity” or “percentage of sequence similarity” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Proc Natl Aca Sci USA 87: 2267-2268, 1990; Nucl Acids Res 25: 3389-3402, 1997). The BLAST programs identify homologous sequences by identifying similar segments, which are referred to herein as “high-scoring segment pairs”, between a query amino acid or nucleic acid sequence and a test sequence which is preferably obtained from a protein or nucleic acid sequence database. Preferably, the statistical significance of a high-scoring segment pair is evaluated using the statistical significance formula from Proc Natl Aca Sci USA 87: 2267-2268, 1990, the disclosure of which is incorporated by reference in its entirety. The BLAST programs can be used with the default parameters or with modified parameters provided by the user.

TABLE 1
Insulin nucleic acid sequences

| | NCBI | |
|---|---|---|
| | Reference | |
| Animal | Sequence | Gene Sequence |
| Rat | NC_051336.1 | AACCCTAAGTGACCAGCTACAATCATAGACCATCAGCAAGCAGGTATGTACT |
| | | CTCCTGGGTGAGCCCGGTTCCCCCAGCCAAAACTCTAGGGACTTTAGGAAGGA |
| | | TGTGGGTTCCTCTCTTACATGGACCTTTTCCTAGCCTCAACCCTGCCTATCTTC |
| | | CAGGTCATTGTTCCAACATGGCCCTGTGGATGCGCTTCCTGCCCCTGCTGGCC |
| | | CTGCTCGTCCTCTGGGAGCCCAAGCCTGCCCAGGCTTTTGTCAAACAGCACCT |
| | | TTGTGGTCCTCACCTGGTGGAGGCTCTGTACCTGGTGTGTGGGGAACGTGGTT |
| | | TCTTCTACACACCCAAGTCCCGTCGTGAAGTGGAGGACCCGCAAGTGCCACAA |
| | | CTGGAGCTGGGTGGAGGCCCGGAGGCCGGGGATCTTCAGACCTTGGCACTGG |
| | | AGGTTGCCCGGCAGAAGCGTGGCATTGTGGATCAGTGCTGCACCAGCATCTGC |
| | | TCCCTCTACCAACTGGAGAACTACTGCAACTGAGTCCACCACTCCCCGCCCAC |
| | | CCCTCTGCAATGAATAAAGCCTTTGAATGAGCACCAAAA (SEQ ID NO: 10) |
| Cat | NC_018732.3 | GCCGGCGGGGCCCAGCAGCCAGCAGCCCCCCCCCCCGGGACCACCTGCACCC |
| | | TGACACGGACGGCAAGCAGGTCTGTCCCCACGGGCTCCCCGTCAGCTGAGTCC |
| | | AGGGTGTCACACGGGGCCCCGGGCCCGGGCTCTCGAGCCGGCGGGAGGACGA |
| | | GGGCTCCCAGCACAGAGCATCTGGGGGGCGAGCGCAGGGCGGGGGTCCTGGG |
| | | TGCGGCGCGTGCCCCTCGCCGGCCTCAACCCTGCCCGTCCCCCAGGTCGCTGT |
| | | CCCCCGCCATGGCCCCGTGGACGCGCCTCCTGCCCCTGCTGGCGTTGCTGTCC |
| | | CTCTGGATCCCTGCCCCGACCCGAGCCTTCGTCAACCAGCACCTGTGCGGCTC |
| | | CCACCTGGTGGAGGCGCTGTACCTGGTGTGCGGGGAGCGCGGCTTCTTCTACA |
| | | CGCCCAAGGCCCGCCGGGAGGCGGAGGACCTCCAGGGTGAGCCCCCGCTGCC |
| | | CCCGCTAACCCCACCCCGGCTTCCCGCCTGGCGCCCCGGGGGAATCGGGCCGG |
| | | AGTTTTAAAAAAGAAAAACCACATTTCCCCTGGTGACATCCCGAAAGGCCCG |
| | | CGTCGGGCACCGAGGGCCCCCGTGTGGGCTCCTGCCCTGGCCCGGCCCTGCGG |
| | | CCCGGGAGCCTGCGGCGGGGTGGGCGCCCCACGGCCGGCACGCGGGCGGGCG |
| | | GGCGGGCGGGCGGGCGGGCGGGCGGGAGGCGCGGTCTGTCCCTGAGCGGCCG |
| | | TCCGCCTTGCTGCCCCGCAGGGAAGGACGCGGAGCTGGGGGAGGCGCCTGGC |
| | | GCCGGCGGCCTGCAGCCCTCGGCCCTGGAGGCGCCCCTGCAGAAGCGGGGCA |
| | | TCGTGGAGCAATGCTGTGCCAGCGTCTGCTCGCTGTACCAGCTGGAGCATTAC |
| | | TGCAACTAGAGGGCGCCCGGAGCCCGCCGCCCCTGCGCCCCAACCCGTCCAA |
| | | TAAACCCTTGAACGAGC (SEQ ID NO: 11) |
| Dog | NC_051822.1 | CACCCCGACACGGCCGGCAAACAGGTCTGTCCCCACGGGCTCCCCGCCGCCG |
| | | CCTCCCCGCCAGCCTGTGCTCTCAAGGCAGCAGGAGGAGAAGAGCTGCCTCG |
| | | GGGCCTTTGCGGGGTGGGCTCAGGGTGGGGGGGGCCGTGCCCCTTGCCAGCC |
| | | TCAACCCTGCCTGTCCCCAGGTCGCCATGGCCCTCTGGATGCGCCTCCTGCCC |
| | | CTGCTGGCCCTGCTGGCCCTCTGGGCGCCCGCGCCCACCCGAGCCTTCGTTAA |
| | | CCAGCACCTGTGTGGCTCCCACCTGGTAGAGGCTCTGTACCTGGTGTGCGGGG |
| | | AGCGCGGCTTCTTCTACACGCCTAAGGCCCGCCGGGAGGTGGAGGACCTGCA |
| | | GGGTAAGCCCCCGCCGCCCCCGCCGCCCCCGCCCTGGCTCCCTACCTGGCCCC |
| | | AGGGGCAGGCCAGGTGGAAATATTAAAAAGAAAAATGACTTTCCCTTGGTCT |
| | | ACATCCTGCAAGGGACCAGCTCCTTGGTCAGGGTCGGGCACCAAAAGCCTGA |
| | | GGGCAGCCTCCCACCTTGGCACCACCCTGGGGCCTGGGAGCCACTGGCACGG |
| | | GGTGGGGTGGGCGGGGCGCGCTCTCTCCCTGACCCTGACCCGCTCTCCGCCTG |
| | | GCTCCTCCGCAGTGAGGGACGTGGAGCTGGCCGGGGCGCCTGGCGAGGGCGG |
| | | CCTGCAGCCCCTGGCCCTGGAGGGGGCCCTGCAGAAGCGAGGCATCGTGGAG |
| | | CAGTGCTGCACCAGCATCTGCTCCCTCTACCAGCTGGAGAATTACTGCAACTA |
| | | GGGGCGCGGGGGGCAGGACGTGGCAGCACCTGCTGCAGGTCACGGTGGCCGC |
| | | AAGCCTTCGGCTCTCTGCACCCCAAGTGATTCAATAAACCCTCTGAATG (SEQ |
| | | ID NO: 12) |
| Human | NC_000011.10 | AGCCCTCCAGGACAGGCTGCATCAGAAGAGGCCATCAAGCAGGTCTGTTCCA |
| | | AGGGCCTTTGCGTCAGGTGGGCTCAGGATTCCAGGGTGGCTGGACCCCAGGC |
| | | CCCAGCTCTGCAGCAGGGAGGACGTGGCTGGGCTCGTGAAGCATGTGGGGGT |
| | | GAGCCCAGGGGCCCCAAGGCAGGGCACCTGGCCTTCAGCCTGCCTCAGCCCT |
| | | GCCTGTCTCCCAGATCACTGTCCTTCTGCCATGGCCCTGTGGATGCGCCTCCTG |
| | | CCCCTGCTGGCGCTGCTGGCCCTCTGGGGACCTGACCCAGCCGCAGCCTTTGT |
| | | GAACCAACACCTGTGCGGCTCACACCTGGTGGAAGCTCTCTACCTAGTGTGCG |
| | | GGGAACGAGGCTTCTTCTACACACCCAAGACCCGCCGGGAGGCAGAGGACCT |
| | | GCAGGGTGAGCCAACTGCCCATTGCTGCCCCTGGCCGCCCCCAGCCACCCCCT |
| | | GCTCCTGGCGCTCCCACCCAGCATGGGCAGAAGGGGGCAGGAGGCTGCCACC |
| | | CAGCAGGGGGTCAGGTGCACTTTTTTAAAAAGAAGTTCTCTTGGTCACGTCCT |
| | | AAAAGTGACCAGCTCCCTGTGGCCCAGTCAGAATCTCAGCCTGAGGACGGTG |
| | | TTGGCTTCGGCAGCCCCGAGATACATCAGAGGGTGGGCACGCTCCTCCCTCCA |
| | | CTCGCCCCTCAAACAAATGCCCCGCAGCCCATTTCTCCACCCTCATTTGATGA |
| | | CCGCAGATTCAAGTGTTTTGTTAAGTAAAGTCCTGGGTGACCTGGGGTCACAG |
| | | GGTGCCCCACGCTGCCTGCCTCTGGGCGAACACCCCATCACGCCCGGAGGAG |
| | | GGCGTGGCTGCCTGCCTGAGTGGGCCAGACCCCTGTCGCCAGGCCTCACGGCA |
| | | GCTCCATAGTCAGGAGATGGGGAAGATGCTGGGGACAGGCCCTGGGGAGAAG |
| | | TACTGGGATCACCTGTTCAGGCTCCCACTGTGACGCTGCCCCGGGGCGGGGGA |
| | | AGGAGGTGGGACATGTGGGCGTTGGGGCCTGTAGGTCCACACCCAGTGTGGG |
| | | TGACCCTCCCTCTAACCTGGGTCCAGCCCGGCTGGAGATGGGTGGGAGTGCGA |
| | | CCTAGGGCTGGCGGGCAGGCGGGCACTGTGTCTCCCTGACTGTGTCCTCCTGT |
| | | GTCCCTCTGCCTCGCCGCTGTTCCGGAACCTGCTCTGCGCGGCACGTCCTGGC |
| | | AGTGGGGCAGGTGGAGCTGGGCGGGGGCCCTGGTGCAGGCAGCCTGCAGCCC |
| | | TTGGCCCTGGAGGGGTCCCTGCAGAAGCGTGGCATTGTGGAACAATGCTGTAC |
| | | CAGCATCTGCTCCCTCTACCAGCTGGAGAACTACTGCAACTAGACGCAGCCCG |
| | | CAGGCAGCCCCACACCCGCCGCCTCCTGCACCGAGAGAGATGGAATAAAGCC |
| | | CTTGAACCAGC (SEQ ID NO: 13) |

The constructs of the present invention also comprise an albumin 3′ untranslated region (UTR). As used herein, the term “albumin 3′ UTR” refers to either the full-length albumin 3′ UTR sequence or a functional portion thereof. The albumin 3′ UTR is known to contribute to the longevity of albumin mRNA in hepatocytes. Thus, inclusion of this component in the constructs of the present invention increases protein production. In certain embodiments, the albumin 3′ UTR is SEQ ID NO:8, i.e., the albumin 3′ UTR utilized in the C36-AAV8 construct (SEQ ID NO:1). The inventors obtained this sequence from an expression vector plasmid from Mirus (pMIR0375), but this sequence can also be chemically synthesized or amplified via PCR using reverse transcribed mRNA from the liver.

### Host Cells, Viral Particles, and Packaging Cell Lines

In a second aspect, the present invention provides host cells transduced with the constructs described herein. As used herein, the term “host cell” refers to any prokaryotic or eukaryotic cell that contains a construct of the present invention. This term also includes cells that have been genetically engineered such that a construct of the present invention is integrated into its genome.

In a third aspect, the present invention provides recombinant adeno-associated virus serotype 8 (AAV8) virus particles comprising the constructs described herein. To generate such particles, the constructs of the present invention are cloned into an AAV8 vector backbone. Viral particles may then be generated by helper virus-free co-transfection of HEK 293T cells with three plasmids: (1) an AAV8 vector comprising a construct of the present invention, (2) a packaging plasmid carrying the AAV rep (packaging) and cap (structural) genes (i.e., pAAV2/8), and (3) a helper plasmid carrying the AAV helper functions. For a detailed description of viral production methods, see Ayuso et al. (Gene Ther 17(4):503-10, 2010), which is hereby incorporated by reference in its entirety. Other suitable methods for producing AAV8 virus particles and well known and understood in the art.

As used herein, the term “virus particle” is used to refer to a virion consisting of nucleic acid surrounded by a protective protein coat called a capsid. The term “viral vector” is used to describe a virus particle that is used to deliver genetic material (e.g., the constructs of the present invention) into cells. The shorthand “AAV vector” or “AAV8 vector” is commonly used to refer to a viral vector in the art. A “recombinant viral vector” is a viral vector that has been genetically manipulated. For example, the recombinant AAV8 vectors of the present invention have been engineered to deliver a heterologous polypeptide encoding insulin to a subject in need thereof.

In a fourth aspect, the present invention provides packaging cell lines for producing the virus particles described herein. As used herein, the term “packaging cell line” is used to refer to a cell line that provides all the proteins necessary for AAV virus production and maturation. Suitable packaging cell lines for use with the present invention include, without limitation, HEK 293T cells and HEK 293 cell variants. The packaging cell line should be selected with the method of viral production in mind. For example, cells that have strong adhesion properties should be selected for growth in culture plates, whereas cells lacking adhesion properties should be selected for growth in suspension culture. In some embodiments, the packaging cell line comprises the complement of any genes that have been functionally deleted in the virus particle that it is used to produce the virus, thus allowing for replication incompetent viral particles to be produced (e.g., the virus can be produced in the packaging cell line, enter a target cell, and express the protein of interest encoded by the viral construct).

### Methods of Treatment

In a fifth aspect, the present invention provides methods for controlling blood glucose levels in a mammal by administering the recombinant AAV8 virus particles described herein. In these methods, the mammal's glucose levels are controlled by the glucose-regulated synthesis of insulin from the nucleic acid construct. Insulin expression controls blood glucose levels by signaling to liver, muscle, and fat cells to take in glucose from the blood.

By “controlling glucose levels” we mean that the methods result in glucose regulation wherein post-prandial spikes in glucose levels return to a normal acceptable range commensurate with that of a non-diabetic within a reasonable period of time. In non-diabetic individuals, the blood glucose returns to normal at about 2 hours post meal. As is demonstrated in the Examples, following treatment with the virus particles of the present invention, the serum insulin levels increased soon after the increase in blood glucose levels, but the insulin levels did not stay high for too long and followed the blood glucose level curve with a delay of about 15-30 minutes. These results demonstrate that use of these methods enables the liver cells of the subject to synthesize and secrete insulin in response to changing glucose levels. Typically, the methods allow blood glucose levels to stay within 80-180 mg/dl after treatment, preferably within 80-150 mg/dl which is considered “suitable glucose regulation”. For reference, levels from 80-140 mg/dl are considered normal, and levels from 60-180 mg/dl are considered adequate for gene therapy control. The high end of this range results from a temporary post meal spike. Thus, if the glucose concentration does rise above 150 mg/dl, it will not stay at that level for more than a short period of time (30-60 minutes). In preferred embodiments, the methods provide suitable glucose regulation that lasts longer than a month, longer than six months, or longer than a year. Ideally, the methods provide suitable glucose regulation that lasts for multiple years.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent. In some embodiments, the virus particle is administered by vascular injection. The high affinity of AAV8 for the liver and the use of a liver-specific promoter (e.g., the albumin promoter) should ensure that the gene therapy is liver specific. In certain embodiments, the virus particle is injected into the femoral artery, which should increase uptake into the liver.

In some embodiments, the virus particle is administered with a pharmaceutically acceptable carrier. “Pharmaceutically acceptable carriers” are known in the art and include, but are not limited to, diluents, preservatives, solubilizers, emulsifiers, liposomes, nanoparticles, and adjuvants. Pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, and suspensions, including saline and buffered media.

Ideally, the virus particles are administered in a therapeutically effective amount. The term “therapeutically effective amount” refers to an amount sufficient to effect beneficial or desirable biological or clinical results. In the present methods, a therapeutically effective amount is an amount that is sufficient to control glucose levels in a mammal, as discussed above. Methods for determining an effective means of administration and dosage are well known to those of skill in the art and will vary with the formulation used for therapy and the subject (e.g., species, age, health, etc.) being treated. Single or multiple administrations can be carried out with the dose level and pattern being selected by the treating physician. In some embodiments, the virus particle is administered at a dose of 1×10¹⁵ vector genome/kg (vg/kg) or lower, preferably about 1×10¹² vg/kg or lower.

In some embodiments, the methods further comprise measuring the mammal's insulin levels. This can be accomplished using, for example, an enzyme-linked immunosorbent assay (ELISA), radioimmunoassay, or immunoprecipitation followed by high-performance liquid chromatography (HPLC) or mass spectrometry (MS). When one observes the mammal's blood glucose and/or insulin levels after treatment, the mammal's blood glucose and/or insulin levels should be controlled and normal.

The methods of the present invention involve the treatment of a mammal. In certain embodiments, the mammal is a rat, dog, or cat. In certain embodiments, the mammal is a human.

The constructs and virus particles disclosed herein are designed to deliver insulin-encoding polynucleotides to the liver. These gene therapies enable the liver cells of patients with type I diabetes to synthesize and secrete insulin in response to changing glucose levels. Thus, in some embodiments, the mammal has type I diabetes. Thus, the virus particles are administered in vivo to the mammal and delivered to the liver wherein the polynucleotide construct can express insulin encoded in the polynucleotide.

In a related aspect, the present invention provides methods of transducing hepatocytes with the virus particles of the present invention ex vivo. In these embodiments, the constructs of the present invention are delivered into isolated hepatocytes via transduction with recombinant AAV8 virus particles to achieve glucose-regulated expression of insulin in the hepatocytes. For example, the hepatocytes may be harvested from a patient through a biopsy, expanded in cell culture, and transduced with a construct of the present invention. These methods may further comprise transplanting an appropriate number of transduced hepatocytes into a mammal as to provide the necessary amount of insulin. Transplantation may be performed, for example, using radiological and ultrasound guidance.

### Methods of Viral Production

In a sixth aspect, the present invention provides methods of producing an AAV8 virus particle. The methods comprise (a) transducing a host cell with: (i) a plasmid comprising SEQ ID NO:1, (ii) a packaging plasmid, and (iii) a helper plasmid; (b) collecting the supernatant and cells from the culture after a suitable time; and (c) isolating the virus. Suitable amounts of time to culture the cells are known in the art and include, for example, at least 48 hours, at least 72 hours, or more.

Virus can be isolated from the supernatant and from lysed cells by methods known and understood in the art. In some embodiments, the methods further comprise purifying or isolating the virus from the supernatant or lysed cells. Suitable methods for purifying or isolating virus from cell culture include, but are not limited to, cesium chloride density gradient centrifugation and affinity purification (e.g., using a porous matrix modified to retain the virus).

The terms “transduced,” “transfected,” and “transformed” all refer to processes by which an exogenous nucleic acid is introduced into a host cell. The term “transduced” specifically refers to the process by which a virus transfers a nucleic into a host cell.

The term “packaging plasmid” refers to a plasmid that encodes components of the viral capsid. For AAV production, the packaging plasmid may encode the AAV genes rep and cap. In some embodiments, the packaging plasmid is pAAV2/8. The term “helper plasmid” refers to a plasmid that encodes adenovirus helper functions. Proteins encoded by all three plasmids that are transduced into the host cell in the present methods are required for viral replication, as is well known in the art.

In some embodiments, the methods further comprise concentrating the virus. Suitable methods for concentrating virus include, but are not limited to, ultracentrifugation and dialysis.

In some embodiments, the methods further comprise dialyzing the supernatant. For some applications, it may be advantageous to replace the cell culture media present in the supernatant with a solution that is better for long-term storage. Suitable solutions for storage include, but are not limited to, phosphate-buffered saline (PBS), PBS with plutonic acid, saline adjusted to pH 7-7.4 with or without pluronic acid (0.001-0.01%), and Ringer's lactate solution. However, any biocompatible, osmotically balanced, neutral pH fluid should be suitable for storage.

In the Examples, the inventors demonstrate that using a single GIRE in their insulin-encoding nucleic acid constructs increased construct production by about 10-fold. Thus, in some embodiments, the methods produce high yields of AAV8 virus particles. In certain embodiments, the methods yield greater than 1×10⁹ vector genomes per cm² of cell culture plate, preferably about 3.1×10⁹ vector genomes per cm² of cell culture plate, for cells grown in adherent cell culture. However, the yield will vary due to several factors, including the level of confluency at the time of plasmid co-transfection, etc.

It should be apparent to those skilled in the art that many additional modifications besides those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

The present invention has been described in terms of one or more preferred embodiments, and it should be appreciated that many equivalents, alternatives, variations, and modifications, aside from those expressly stated, are possible and within the scope of the invention.

The invention will be more fully understood upon consideration of the following non-limiting examples.

### EXAMPLES

In previous work, we have made many gradual, incremental improvements to our insulin gene therapy constructs. Some of our recently developed insulin constructs are depicted in FIG. 1. In most cases, the modifications made to the constructs resulted in an outcome that could be explained. However, when we reduced the number of glucose inducible regulatory elements (GIREs) from three units to one unit in the insulin gene construct sc.C19-AAV8 (forming the new construct C36-AAV8) we achieved unexpected and remarkable improvements.

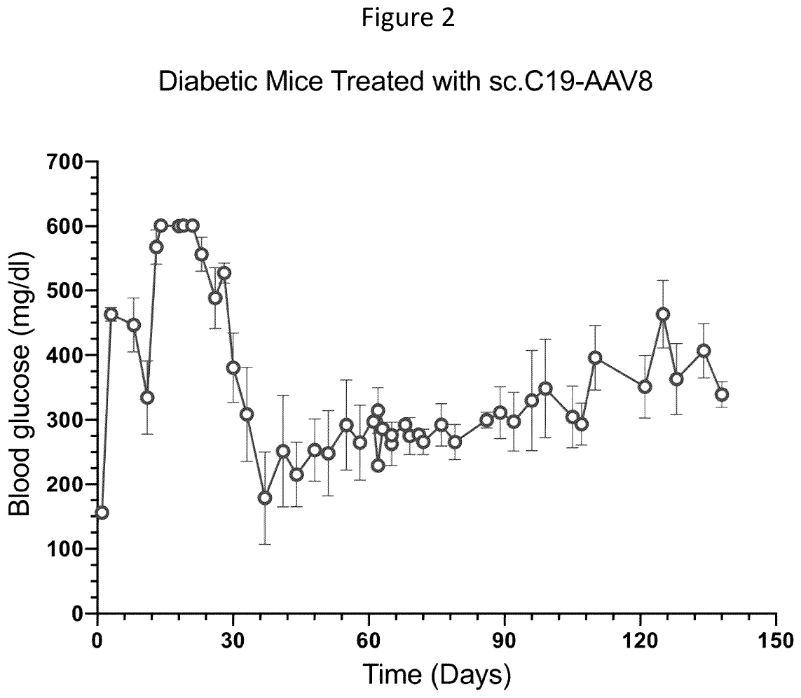

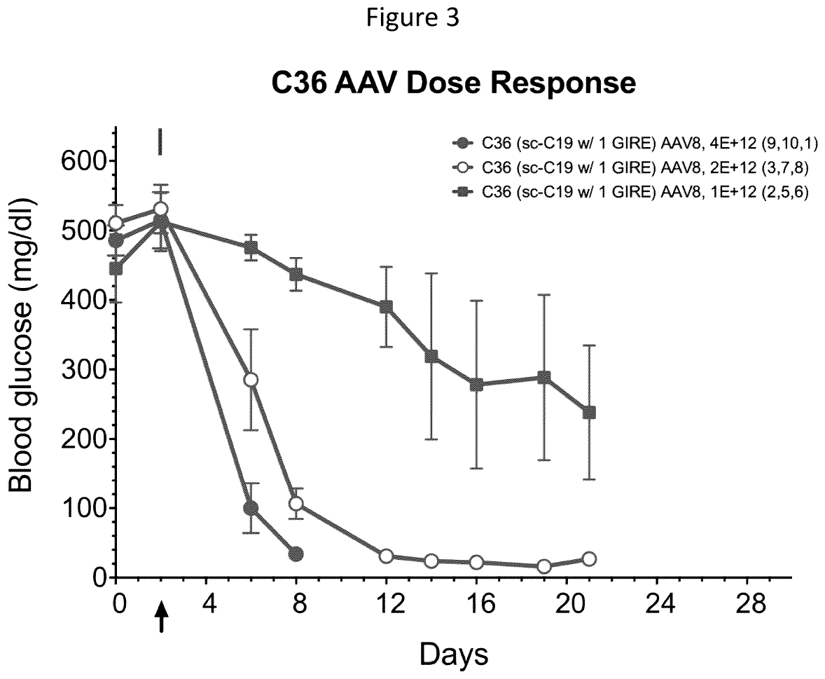

First, the in vivo therapeutic efficacy of C36-AAV8 (i.e., the construct with a single GIRE) was significantly higher than that of sc.C19-AAV8 (i.e., the construct with three GIREs). sc.C19-AAV8 required a dose of 8×10¹² vg/kg to correct diabetic hyperglycemia less than adequately (FIG. 2), whereas C36-AAV8 had an in vivo efficacy that was approximately 8× greater (FIG. 3). C36-AAV8 was tested at three different doses: 4×10¹² vg/kg, 2×10¹² vg/kg, and 1×10¹² vg/kg. All animals receiving the two higher doses (i.e., 4×10¹² vg/kg and 2×10¹² vg/kg) became hypoglycemic relatively quickly and either died or had to be humanely euthanized. The animals treated at the lowest dose (i.e., 1×10¹² vg/kg) corrected hyperglycemia slowly, showing a significant reduction in blood glucose from over 600 mg/dl to 200-300 mg/dl. We expect that either this dose or a slightly lower dose would be optimum.

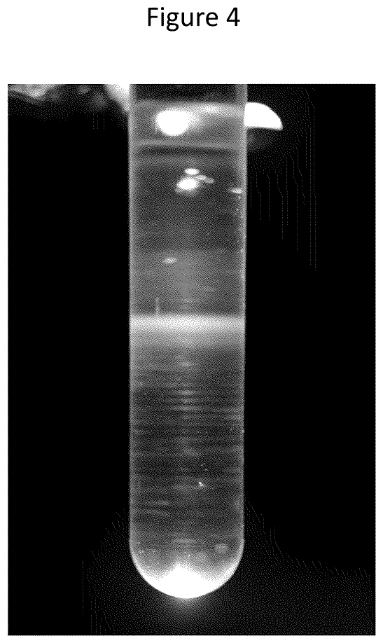

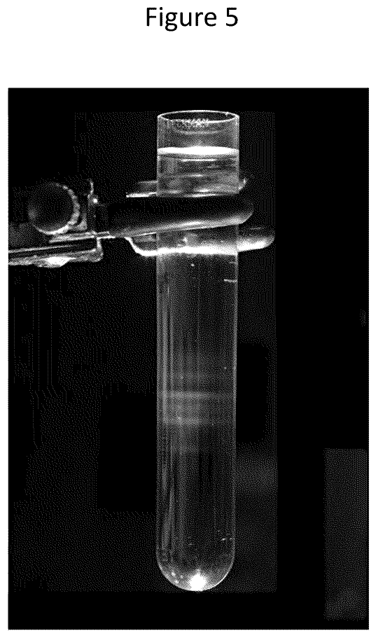

Second, the viral yield of C36-AAV8 (i.e., the construct with a single GIRE) was drastically increased over that of sc.C19-AAV8 (i.e., the construct with three GIREs). Viral particles were generated by helper virus-free co-transfection of HEK 293T cells with three plasmids: (1) a vector comprising the C36-AAV8 construct (SEQ ID NO:1), (2) a packaging plasmid carrying the AAV rep and cap genes (i.e., pAAV2/8), and (3) a helper plasmid carrying the AAV helper functions. Virus was purified using a cesium chloride (CsCl) density gradient. For a detailed description of the viral production method, see Ayuso et al. (Gene Ther 17(4):503-10, 2010). In the C36-AAV8 density gradient, we observed a single major band containing functionally active AAV8 along with a minor, slightly higher density, lower band that could be readily separated from the major band (FIG. 4). Thus, the C36-AAV8 construct predominantly yielded biologically active AAV8. The sc.C19-AAV8 construct showed a different profile that is obvious with the naked eye. In the sc.C19-AAV8 density gradient, we observed multiple, closely spaced, weaker bands in the CsCl density gradient of which only one (i.e., the second heaviest density band) contained the major biological activity (FIG. 5). When we quantitated the viral yield of both AAV8 preparations, we found that the yield of C36-AAV8 was approximately 10-fold greater than the yield of sc.C19-AAV8. Specifically, in a standard production batch using 42 15 cm diameter plates of adherent HEK 293T cells, the yield of C36-AAV8 was 5.61×10¹³ viral genomes, whereas the yield of sc.C19-AAV8 was 5.95×10¹² viral genomes (multiple batches). Thus, as demonstrated, the C36-AAV8 construct predominantly yielded biologically active AAV8 as compared to other constructs.

Collectively, these two improvements associated with the C36-AAV8 construct offer (1) an approximately 80-fold reduction in the cost of producing a gene therapy treatment, and (2) a significant improvement in treatment safety. The fact that C36-AAV8 can provide a comparable therapeutic benefit at a dosage that is 4-8-fold lower than the dosage required for sc.C19-AAV8 is significant in that it dramatically reduces the risk of immunological response to AAV8, making the use of this construct a far safer option for treating diabetes.

What is claimed:

1. A nucleic acid construct for producing a recombinant AAV8 vector encoding insulin comprising:
a) a 5′ inverted terminal repeat (ITR);
b) a promoter enhancer;
c) a glucose inducible regulatory element (GIRE);
d) a liver-specific promoter;
e) a translational enhancer;
f) a polynucleotide encoding insulin with a modified peptidase site;
g) an albumin 3′ untranslated region (UTR); and
h) a 3′ ITR;
wherein the vector contains only one GIRE.

2. The construct of claim 1, wherein the 5′ ITR is SEQ ID NO:2.

3. The construct of claim 1 or 2, wherein the promoter enhancer is an alpha-fetoprotein enhancer.

4. The construct claim 3, wherein the alpha-fetoprotein enhancer is SEQ ID NO:3.

5. The construct of any one of the preceding claims, wherein the GIRE is SEQ ID NO:4.

6. The construct of any one of the preceding claims, wherein the liver-specific promoter is an albumin promoter.

7. The construct claim 6, wherein the albumin promoter is SEQ ID NO:5.

8. The construct of any one of the preceding claims, wherein the translational enhancer is a vascular endothelial growth factor (VEGF) translational enhancer.

9. The construct claim 8, wherein the VEGF translational enhancer is SEQ ID NO:6.

10. The construct of any one of the preceding claims, wherein the polynucleotide encoding insulin comprises a furin cleavage site and can be processed by furin.

11. The construct claim 10, wherein the polynucleotide encoding insulin encodes rat insulin, dog insulin, or cat insulin.

12. The construct claim 11, wherein the polynucleotide encoding insulin is SEQ ID NO:7.

13. The construct claim 10, wherein the polynucleotide encoding insulin encodes human insulin.

14. The construct of any one of the preceding claims, wherein the albumin 3′ UTR is SEQ ID NO:8.

15. The construct of any one of the preceding claims, wherein the 3′ ITR is SEQ ID NO:9.

16. The construct of any one of the preceding claims, wherein components (a)-(h) are present in the vector in the following 5′ to 3′ order: (a), (b), (c), (d), (e), (f), (g), and (h).

17. The construct of any one of the preceding claims, wherein the construct is SEQ ID NO:1.

18. A host cell transduced with the construct of any one of claims 1-17.

19. A recombinant adeno-associated virus serotype 8 (AAV8) virus particle comprising the construct of any one of claims 1-17.

20. A packaging cell line for producing the virus particle of claim 19.

21. The packaging cell line of claim 20, wherein the cell line comprises the complement of any genes functionally deleted in the virus particle of claim 19.

22. A method for controlling blood glucose levels in a mammal by administering the recombinant AAV8 virus particle of claim 19, wherein the mammal's glucose levels are controlled by the glucose-regulated synthesis of insulin from the nucleic acid construct.

23. The method of claim 22, wherein the method further comprises measuring the mammal's insulin levels.

24. The method of claim 22 or 23, wherein the virus particle is administered by vascular injection.

25. The method of any one of claims 22-24, wherein the virus particle is administered at a dose of about 1×10¹⁰ to about 1×10¹⁵ vector genomes/kilogram (vg/kg), preferably about 1×10¹⁰ to about 1×10¹³ vg/kg.

26. The method of any one of claims 22-25, wherein the mammal has type I diabetes.

27. The method of any one of claims 22-26, wherein the mammal is a rat, dog, or cat.

28. The method of any one of claims 22-26, wherein the mammal is a human.

29. A method of producing an AAV8 virus particle, the method comprising:
a) transducing a host cell with:
i. a plasmid comprising SEQ ID NO:1,
ii. a packaging plasmid, and
iii. a helper plasmid;
b) collecting the supernatant and the cells from culture after a suitable time; and
c) isolating virus particles.

30. The method of claim 29, further comprising purifying the virus.

31. The method of claim 29 or 30 further comprising concentrating the virus.

32. The method of any one of claims 29-31 further comprising dialyzing the supernatant.

33. The method of any one of claims 29-32, wherein the method yields greater than 1×10⁹ vector genomes per cm² of cell culture plates for cells grown in adherent cell culture.


SEQUENCE LISTING (14 sequences)

SEQ ID NO: 1 - DNA, 5925 bp, Artificial Sequence
  Synthetic - C36-AAV8 construct
cagctgcgcgctcgctcgctcactgaggccgcccgggcaaagcccgggcgtcgggcgacctttggtcgcccggcctcagtgagcgagcgagcgcgcagagagggagtggggttcctgctattcccatatgattctagagctcgtcgacccgcccccttcaccaagatctttttgatggcagagttcagtttaccgggtcacattgtacctgggaagattcaaggatttatggaaaaagtcaacaacaggagtcagagcagccggaaaagcatggactctgtacttaggactgcgctttgagcaatggcacagcaagctttaaccctgtttgcagtcagcacacaaactgtggttcaaagctccactttatctcttcttgtggaattcagatatcagatcagtttaaaccttgcggccgccagttctcacgtggtggccacgtgcttgggcactctagtgctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaataatctacagttattggttaaagaagtatattagagcgagtctttctgcacacagatcaccttcctatcaaccccactagcctctggcaaaggtaccagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggctctgtggatgagattcctgcctctgctggccctgctggtgctgtgggaacctaagcctgcccaggccttcgtgaagcagcacctgtgtggaccccacctggtggaagccctgtacctcgtgtgtggcgagagaggcttcttctacacccctaggaccaagagagaggtggaagatccccaggtgccccagctggaactgggcggaggacctgaagctggcgacctgcagacactggccctggaagtggccagacagaaaaggggcatcgtggaccagtgctgcaccagcatctgcagcctgtatcagctggaaaactactgcaactgaacgcagcctgcaggcagcgtcgagtaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctatcgattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaaccatggtataggtaaggaatataaaacatggcttttaccttagaaaaaacaattctaaaattcatatgattctagaactagtggggtaccgaattcagatcgagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 2 - DNA, 109 bp, Artificial Sequence
  Synthetic - mutant 5′ inverted terminal repeat (ITR)
cagctgcgcgctcgctcgctcactgaggccgcccgggcaaagcccgggcgtcgggcgacctttggtcgcccggcctcagtgagcgagcgagcgcgcagagagggagtgg

SEQ ID NO: 3 - DNA, 251 bp, Artificial Sequence
  Synthetic - Alfa-fetoprotein enhancer
aagatctttttgatggcagagttcagtttaccgggtcacattgtacctgggaagattcaaggatttatggaaaaagtcaacaacaggagtcagagcagccggaaaagcatggactctgtacttaggactgcgctttgagcaatggcacagcaagctttaaccctgtttgcagtcagcacacaaactgtggttcaaagctccactttatctcttcttgtggaattcagatatcagatcagtttaaaccttgc

SEQ ID NO: 4 - DNA, 17 bp, Artificial Sequence
  Synthetic - glucose inducible regulatory element (GIRE) from S14
cacgtggtggccacgtg

SEQ ID NO: 5 - DNA, 327 bp, Artificial Sequence
  Synthetic - Rat albumin promoter
ctagtgctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaataatctacagttattggttaaagaagtatattagagcgagtctttctgcacacagatcaccttcctatcaaccccactagcctctggcaaaggtac

SEQ ID NO: 6 - DNA, 164 bp, Artificial Sequence
  Synthetic - Vascular endothelial growth factor (VEGF) translational enhancer
cagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaacc

SEQ ID NO: 7 - DNA, 333 bp, Artificial Sequence
  Synthetic - Polynucleotide encoding rat insulin 1, furin cleavable, codon optimized
atggctctgtggatgagattcctgcctctgctggccctgctggtgctgtgggaacctaagcctgcccaggccttcgtgaagcagcacctgtgtggaccccacctggtggaagccctgtacctcgtgtgtggcgagagaggcttcttctacacccctaggaccaagagagaggtggaagatccccaggtgccccagctggaactgggcggaggacctgaagctggcgacctgcagacactggccctggaagtggccagacagaaaaggggcatcgtggaccagtgctgcaccagcatctgcagcctgtatcagctggaaaactactgcaactga

SEQ ID NO: 8 - DNA, 842 bp, Artificial Sequence
  Synthetic - Albumin 3′ UTR
acgcagcctgcaggcagcgtcgagtaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctatcgattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaaccatggtataggtaaggaatataaaacatggcttttaccttagaaaaaacaattctaaaatt

SEQ ID NO: 9 - DNA, 136 bp, Artificial Sequence
  Synthetic - 3′ inverted terminal repeat (ITR)
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcg

SEQ ID NO: 10 - DNA, 568 bp, Rattus norvegicus
aaccctaagtgaccagctacaatcatagaccatcagcaagcaggtatgtactctcctgggtgagcccggttcccccagccaaaactctagggactttaggaaggatgtgggttcctctcttacatggaccttttcctagcctcaaccctgcctatcttccaggtcattgttccaacatggccctgtggatgcgcttcctgcccctgctggccctgctcgtcctctgggagcccaagcctgcccaggcttttgtcaaacagcacctttgtggtcctcacctggtggaggctctgtacctggtgtgtggggaacgtggtttcttctacacacccaagtcccgtcgtgaagtggaggacccgcaagtgccacaactggagctgggtggaggcccggaggccggggatcttcagaccttggcactggaggttgcccggcagaagcgtggcattgtggatcagtgctgcaccagcatctgctccctctaccaactggagaactactgcaactgagtccaccactccccgcccacccctctgcaatgaataaagcctttgaatgagcaccaaaa

SEQ ID NO: 11 - DNA, 961 bp, Felis catus
gccggcggggcccagcagccagcagcccccccccccgggaccacctgcaccctgacacggacggcaagcaggtctgtccccacgggctccccgtcagctgagtccagggtgtcacacggggccccgggcccgggctctcgagccggcgggaggacgagggctcccagcacagagcatctggggggcgagcgcagggcgggggtcctgggtgcggcgcgtgcccctcgccggcctcaaccctgcccgtcccccaggtcgctgtcccccgccatggccccgtggacgcgcctcctgcccctgctggcgttgctgtccctctggatccctgccccgacccgagccttcgtcaaccagcacctgtgcggctcccacctggtggaggcgctgtacctggtgtgcggggagcgcggcttcttctacacgcccaaggcccgccgggaggcggaggacctccagggtgagcccccgctgcccccgctaaccccaccccggcttcccgcctggcgccccgggggaatcgggccggagttttaaaaaagaaaaaccacatttcccctggtgacatcccgaaaggcccgcgtcgggcaccgagggcccccgtgtgggctcctgccctggcccggccctgcggcccgggagcctgcggcggggtgggcgccccacggccggcacgcgggcgggcgggcgggcgggcgggcgggcgggcgggaggcgcggtctgtccctgagcggccgtccgccttgctgccccgcagggaaggacgcggagctgggggaggcgcctggcgccggcggcctgcagccctcggccctggaggcgcccctgcagaagcggggcatcgtggagcaatgctgtgccagcgtctgctcgctgtaccagctggagcattactgcaactagagggcgcccggagcccgccgcccctgcgccccaacccgtccaataaacccttgaacgagc

SEQ ID NO: 12 - DNA, 887 bp, Canis lupus
caccccgacacggccggcaaacaggtctgtccccacgggctccccgccgccgcctccccgccagcctgtgctctcaaggcagcaggaggagaagagctgcctcggggcctttgcggggtgggctcagggtggggggggccgtgccccttgccagcctcaaccctgcctgtccccaggtcgccatggccctctggatgcgcctcctgcccctgctggccctgctggccctctgggcgcccgcgcccacccgagccttcgttaaccagcacctgtgtggctcccacctggtagaggctctgtacctggtgtgcggggagcgcggcttcttctacacgcctaaggcccgccgggaggtggaggacctgcagggtaagcccccgccgcccccgccgcccccgccctggctccctacctggccccaggggcaggccaggtggaaatattaaaaagaaaaatgactttcccttggtctacatcctgcaagggaccagctccttggtcagggtcgggcaccaaaagcctgagggcagcctcccaccttggcaccaccctggggcctgggagccactggcacggggtggggtgggcggggcgcgctctctccctgaccctgacccgctctccgcctggctcctccgcagtgagggacgtggagctggccggggcgcctggcgagggcggcctgcagcccctggccctggagggggccctgcagaagcgaggcatcgtggagcagtgctgcaccagcatctgctccctctaccagctggagaattactgcaactaggggcgcggggggcaggacgtggcagcacctgctgcaggtcacggtggccgcaagccttcggctctctgcaccccaagtgattcaataaaccctctgaatg

SEQ ID NO: 13 - DNA, 1431 bp, Homo sapiens
agccctccaggacaggctgcatcagaagaggccatcaagcaggtctgttccaagggcctttgcgtcaggtgggctcaggattccagggtggctggaccccaggccccagctctgcagcagggaggacgtggctgggctcgtgaagcatgtgggggtgagcccaggggccccaaggcagggcacctggccttcagcctgcctcagccctgcctgtctcccagatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcagggtgagccaactgcccattgctgcccctggccgcccccagccaccccctgctcctggcgctcccacccagcatgggcagaagggggcaggaggctgccacccagcagggggtcaggtgcacttttttaaaaagaagttctcttggtcacgtcctaaaagtgaccagctccctgtggcccagtcagaatctcagcctgaggacggtgttggcttcggcagccccgagatacatcagagggtgggcacgctcctccctccactcgcccctcaaacaaatgccccgcagcccatttctccaccctcatttgatgaccgcagattcaagtgttttgttaagtaaagtcctgggtgacctggggtcacagggtgccccacgctgcctgcctctgggcgaacaccccatcacgcccggaggagggcgtggctgcctgcctgagtgggccagacccctgtcgccaggcctcacggcagctccatagtcaggagatggggaagatgctggggacaggccctggggagaagtactgggatcacctgttcaggctcccactgtgacgctgccccggggcgggggaaggaggtgggacatgtgggcgttggggcctgtaggtccacacccagtgtgggtgaccctccctctaacctgggtccagcccggctggagatgggtgggagtgcgacctagggctggcgggcaggcgggcactgtgtctccctgactgtgtcctcctgtgtccctctgcctcgccgctgttccggaacctgctctgcgcggcacgtcctggcagtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgcaggcagccccacacccgccgcctcctgcaccgagagagatggaataaagcccttgaaccagc

SEQ ID NO: 14 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN